(19) United States
(12) Patent Application Publication
(10) Pub. No.: US 2021/0254048 A1
(43) Pub. Date: Aug. 19, 2021

(54) COMPOSITIONS AND METHODS TO BARCODE BACTERIOPHAGE RECEPTORS, AND USES THEREOF

(71) Applicant: THE REGENTS OF THE UNIVERSITY OF CALIFORNIA, Oakland, CA (US)

(72) Inventors: Vivek K. Mutalik, Albany, CA (US); Denish Piya, El Cerrito, CA (US); Adam P. Arkin, San Francisco, CA (US)

(21) Appl. No.: 17/169,442

(22) Filed: Feb. 6, 2021

Related U.S. Application Data
(60) Provisional application No. 62/971,130, filed on Feb. 6, 2020.

(51) Int. Cl. C12N 15/10; C12N 7/00; C12Q 1/70; A61K 35/76

(52) U.S. Cl. CPC C12N 15/1065; C12N 7/00; C12N 2795/00033; A61K 35/76; C12N 2795/00022; C12Q 1/701

(57) ABSTRACT

The present invention provides for a nucleic acid encoding a bacteriophage genome comprising a unique n-mer barcode inserted in a non-essential location or gene location within the bacteriophage genome, or a bacteriophage comprising the nucleic acid thereof

### CROSS-REFERENCE TO RELATED APPLICATIONS

This application claims priority to U.S. Provisional Patent Application Ser. No. 62/971,130, filed on Feb. 6, 2020, which is hereby incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of engineered bacteriophages.

### BACKGROUND OF THE INVENTION

Increasing incidents of multidrug resistant bacteria and decrease in the development of new antibiotics have resulted in a global public health concern prompting scientists to seek alternative therapies (Ventola, 2015). Bacteriophages (phages), which infect specific bacterial strains, have been suggested as potential agents to combat this growing threat of multidrug resistant bacterial pathogenesis. Currently, phages are approved by Food and Drug Administration (FDA) for compassionate use only (McCallin et al., 2019) and there have been a few success reports (Schooley et al., 2017, Dedrick et al., 2019). Encouraged by success of sporadic phage therapy, several University affiliated institutions and biotechnology companies have shown interest to conduct clinical trials to make phages commercially available. Besides human application, phages can also beneficial for agricultural applications (Svircev et al., 2018, Hesse & Adhya, 2019). Recent advances in molecular biology techniques have made phage engineering feasible (Pires et al., 2016) and these technologies have been exploited to modify or insert a gene of interest to the phage genome. Unlike naturally occurring phages, these engineered phages are patentable (Todd, 2019; Schmidt, 2019), and there have been some effort in this regard in phage therapy industry (Reardon 2017).

Despite improvements in sequencing technologies, there are many technological gaps that need an urgent attention before we realize the full potential of phage therapy. One of the key challenges that needs attention is to develop methods to quantify and track phages if we hope to make phage therapy a reality. The current methods can be applied to sequence phage genomes in the field applications, but will need substantial investment of money, time and labor to extend it to thousands of samples in diverse environments to track and quantify phages or phage cocktails. As different phages lack any conserved region, each phage formulation need different primer binding regions, sample preparation and sequencing protocols. As phage resistance is common in phage therapy applications, each phage formulation needs to be modified as the resistance develops. Such ‘formulation modifications’ are common in field applications, but there is no standard way to track these changes, quantify the performance of the formulation or individual phages in an economical way. For example, if a particular phage formulation is used in the meat processing plant, there is no way to quantify and track about how the phage formulation is performing. These challenges become seriously limited when we envision in scaling up or cataloguing thousands of different phages available in phage directories. Even though phage biology has achieved a renaissance owing to ongoing antibiotic crisis, most of the experimental techniques applied to quantify phages were developed decades ago (Adams, 1959). Recently, qPCR platform has been developed to quantify phages in a cocktail, but this technique is still low-throughput (Duyvejonck et al., 2019).

By standardizing and unifying the workflows, phage sample or formulation tracking can be carried out economically, with less laborious effort in time efficient manner. One-way to do this is to have identification or artificial genetic tags on each phage such that common sample processing workflows can be established. Identification/artificial genetic tags such as DNA barcodes are inheritable, that are incorporated into an organism's genome but do not confer any phenotypic changes (Block et al., 2004). These barcodes are solely incorporated for easy identification of a particular organism and can be amplified by simple PCR reactions (Block et al., 2004). The primer binding regions can be same for different organisms and have randomized but pre-characterized barcodes that associate the barcodes to different organisms. Here we aim to insert DNA barcodes into phages such that, each barcode identifies its associated phage. There are several advantages of incorporating DNA barcodes to phage genomes. Addition of DNA barcodes to phages is considered genetic manipulation of the organism, which opens an avenue to patent these phages (FIG. 1) (Schmidt, 2019). The barcodes in phage genomes will support multiplex reading of a mixed population (Block et al., 2004), hence they will assist in high-throughput identification of phages in a cocktail or in the environment, following their application. These high-throughput identifications are based on next-generation sequencing techniques, thus facilitating faster turnaround time, with much less laborious sample preparation. These techniques could also serve to check the purity of phage lysates during industry-scale production and cocktail formulation. Barcoded phages also help in keeping track of phages in diverse formulations, in different time course samples to study phage growth/population quantification and helps in adopting the methods when the formulation needs to be changed.

### SUMMARY OF THE INVENTION

The present invention provides for a nucleic acid encoding a bacteriophage genome comprising a unique n-mer barcode inserted in a non-essential location or gene location within the bacteriophage genome, or a bacteriophage comprising the nucleic acid thereof.

In some embodiments, the bacteriophage comprises a wild-type genome, except for the inserted unique n-mer barcode. In some embodiments, the n-mer DNA barcode inserted in a non-essential location or gene location does not interfere with the infection cycle of the bacteriophage, and/or does not compromise the lysis activity and/or growth cycle of a host bacterium infected by the bacteriophage. In some embodiments, the n-mer DNA barcode is flanked by a pair of primer binding regions that bind to a known pair of primers or a pair of primers of known nucleotide sequences, wherein the pair of primer binding regions facilitates the amplification of the n-mer barcode using the known pair of primers or the pair of primers of known nucleotide sequences. The amplification of the n-mer barcode facilitates the determination or identification of the nucleotide sequence or identity of the n-mer barcode.

The present invention provides for a method of identifying the source or origin of a bacteriophage, the method comprising: (a) providing a sample comprises, or is suspected to comprise, a bacteriophage of the present invention; (b) amplifying the n-mer barcode using a known pair of primers or a pair of primers of known nucleotide sequences; (c) determining or identifying the nucleotide sequence of the n-mer barcode; and (d) correlating the n-mer barcode to a known nucleotide sequence which in turns correlates to an identity of a known bacteriophage; such that the source or origin of the bacteriophage is determined based on the correlation obtained in the correlating step.

In some embodiments, the providing step comprises obtaining the sample from a subject. In some embodiments, the subject is a human, such as a human patient suffering or is suspected to be suffering from a disease caused by a bacterium, which the bacteriophage is capable of infecting or is capable of being the host bacterium for the bacteriophage. In some embodiments, the amplifying step comprises performing a polymerase chain reaction (PCR). In some embodiments, the providing step is preceded by one or more of the following steps: constructing the bacteriophage by inserting a unique n-mer barcode into a wild-type bacteriophage, and/or releasing, administering, or selling or transferring the ownership of the bacteriophage, such as administering the bacteriophage to a subject suffering or suspected of suffering from a disease caused by a bacterium, which the bacteriophage is capable of infecting or is capable of being the host bacterium for the bacteriophage.

The present invention provides for a library of bacteriophages wherein each bacteriophage comprises an insertion randomly inserted in the genome of the bacteriophage, such as at least part of the library comprising loss-of-function (LOF) bacteriophages, wherein optionally each bacteriophage comprises an n-mer barcode inserted in a non-essential gene location within the bacteriophage genome comprising loss-of-function (LOF), or a bacteriophage comprising the nucleic acid thereof. In some embodiments, the library is constructed using the RB-Tnseq or CRISPR-Cas system.

The present invention provides for a method of determining the locations with a genome of a bacteriophage wherein the insertion of an n-mer barcode into the genome does not interfere with the infection cycle of the bacteriophage, and/or does not compromise the lysis activity and/or growth cycle of a host bacterium infected by the bacteriophage, the method comprises (a) constructing a library of LOF bacteriophages comprising an insertion randomly inserted the genome of the bacteriophage; (b) determining which bacteriophage is capable of infecting a host bacterium; (c) determining where on the genome of the bacteriophage the insertion is located; (d) inserting a unique n-mer barcode into the non-essential location or gene location identified in the bacteriophage to produce a barcoded bacteriophage; and (e) optionally administering the barcoded bacteriophage to a subject, such as a patient suffering from a disease caused by or infected with a host bacterium that the barcoded bacteriophage is capable of infecting.

The present invention provides for a nucleic acid comprising a bacteriophage genome comprising an n-mer DNA barcode flanked by primer binding region(s) (PBR), wherein the PBR are configured to be useful in amplification of the n-mer DNA barcode, wherein the n-mer DNA barcode comprises a unique randomized or defined DNA barcode.

The present invention provides for a bacteriophage comprised the nucleic acid of the present invention. In some embodiments, the bacteriophage is viable. In some embodiments, the n-mer DNA barcode does not interfere with the infection cycle of the bacteriophage, and/or does not compromise the lysis activity and/or growth cycle of a host bacterium infected by the bacteriophage. In some embodiments, it is easy to amplify the DNA barcode to track and/or analyze bacteriophages. In some embodiments, it is easy to identify, quantify, and/or track the bacteriophage using the DNA barcode.

The present invention provides for use of the bacteriophage and/or use of the library of phages of the present invention in any of the methods disclosed herein, such as those described in FIG. 1.

The present invention provides for a method for screening for gene function for a bacteriophage, the method comprising: (1) (a) providing one or more host organism, such as a species or strain, libraries, (b) providing randomly barcoded transposon sequencing (such as RB-TnSeq), and (c) screening for loss-of-function (LOF) mutant phenotypes; or (2) (a) providing one or more DNA barcoded overexpression strain libraries (such as Dub-seq) using DNA of the host organism and/or phage, and (b) screening for gain-of-function (GOF).

The present invention provides for a method for screening for gene function for a bacteriophage, the method comprising: (a) providing one or more host organism, such as a species or strain, libraries, (b) providing randomly barcoded transposon sequencing (such as RB-TnSeq), and (c) screening for loss-of-function (LOF) mutant phenotypes.

In some embodiments, the providing one or more host organism libraries comprises inserting a barcoded transposon into a host organism, such as using the method taught in Example 1, wherein the host organism(s) can be any host organism, such as any described in Table 1.

TABLE 1
Recent reviews highlights discovery of phage receptors for few model hosts over
the period of decades (Silva et al., FEMS Microbiology letters, 363, 2016, fnw002; Letarov and
Kulikov, Biochemistry (Moscow), 82, 13, 1632-1658, 2017; hereby incorporated by reference in
their entireties)

| Phages | Family | Main host | Receptor(s) |
|---|---|---|---|
| γ | Siphoviridae | Bacillus anthracis | Membrane surface-anchored protein gamma |
| | | | phage receptor (GamR) |
| SPP1 | Siphoviridae | Bacillus subtilis | Glucosyl residues of poly(glycerophosphate) |
| | | | on WTA for reversible binding and |
| | | | membrane protein YueB for irreversible |
| | | | binding |
| ϕ29 | Podoviridae | Bacillus subtilis | Cell WTA (primary receptor) |
| Bam35 | Tectiviridae | Bacillus thuringiensis | N-acetyl-muramic acid (MurNAc) of |
| | | | peptidoglycan in the cell wall |
| LL-H | Siphoviridae | Lactobacillus | Glucose moiety of LTA for reversible |
| | | delbrueckii | adsorption and negatively charged glycerol |
| | | | phosphate group of the LTA for irreversible |
| | | | binding |
| B1 | Siphoviridae | Lactobacillus | Galactose component of the wall |
| | | plantarum | polysaccharide |
| B2 | Siphoviridae | Lactobacillus | Glucose substituents in teichoic acid |
| | | plantarum | |
| 5 | Siphoviridae | Lactococcus lactis | Rhamnose* moieties in the cell wall |
| 13 | | | peptidoglycan for reversible binding and |
| c2 | | | membrane phage infection protein (PIP) for |
| h | | | irreversible binding |
| ml3 | | | |
| kh | | | |
| L | | | |
| φLC3 | Siphoviridae | Lactococcus lactis | Cell wall polysaccharides |
| TP901erm | | | |
| TP901-1 | | | |
| p2 | Siphoviridae | Lactococcus lactis | Cell wall saccharides for reversible |
| | | | attachment and pellicle^{b} |
| | | | phosphohexasaccharide motifs for |
| | | | irreversible adsorption |
| A511 | Myoviridae | Listeria | Peptidoglycan (murein) |
| | | monocytogenes | |
| A118 | Siphoviridae | Listeria | Glucosaminyl and rhamnosyl components of |
| | | monocytogenes | ribitol teichoic acid |
| A500 | Siphoviridae | Listeria | Glucosaminyl residues in teichoic acid |
| | | monocytogenes | |
| φ812 | Myoviridae | Staphylococcus aureus | Anionic backbone of WTA |
| φK | | | |
| 52A | Siphoviridae | Staphylococcus aureus | O-acetyl group from the 6-position of |
| | | | muramic acid residues in murein |
| W | Siphoviridae | Staphylococcus aureus | N-acetylglucosamine (GlcNAc) glycoepitope |
| φ13 | | | on WTA |
| φ47 | | | |
| φ77 | | | |
| φSa2m | | | |
| φSLT | Siphoviridae | Staphylococcus aureus | Poly(glycerophosphate) moiety of LTA |

(a) Receptors that bind RBP of phages

| | | | |
|---|---|---|---|
| φCr30 | Myoviridae | Caulobacter | Paracrystalline surface (S) layer |
| | | crescentus | protein |
| 434 | Siphoviridae | Escherichia coli | Protein 1b (OmpC) |
| BF23 | Siphoviridae | Escherichia coli | Protein BtuB (vitamin B₁₂ receptor) |
| K3 | Myoviridae | Escherichia coli | Protein d or 3A (OmpA) with LPS |
| K10 | Siphoviridae | Escherichia coli | Outer membrane protein LamB |
| | | | (maltodextran selective channel) |
| Me1 | Myoviridae | Escherichia coli | Protein c (OmpC) |
| Mu G(+) | Myoviridae | Escherichia coli | Terminal Glcα-2Glcα1- or |
| | | | GlcNAcα1-2Glcα1- of the LPS |
| Mu G(−) | Myoviridae | Escherichia coli | Termincal glucose with a β1,3 |
| | | | glycosidic linkage |
| | | Erwinia | Terminal glucose linked in β1,6 |
| | | | configuration |
| M1 | Myoviridae | Escherichia coli | Protein OmpA |
| Ox2 | Myoviridae | Escherichia coli | Protein OmpA* |
| ST-1 | Microviridae | Escherichia coli | Terminal Glcα1-2Glcα1- or |
| | | | GlcNAcα1-2Glcα1- of the LPS |
| TLS | Siphoviridae | Escherichia coli | Antibiotic efflux protein TolC and the |
| | | | inner core of LPS |
| Tula | Myoviridae | Escherichia coli | Protein Ia (OmpF) with LPS |
| Tulb | Myoviridae | Escherichia coli | Protein Ib (OmpC) with LPS |
| Tull* | Myoviridae | Escherichia coli | Protein Il* (OmpA) with LPS |
| T1 | Siphoviridae | Escherichia coli | Proteins TonA (FhuA, involved in |
| | | | ferrichrome uptake) and TonB^{b} |
| T2 | Myoviridae | Escherichia coli | Protein Ia (OmpF) with LPS and the |
| | | | outer membrane protein FudL |
| | | | (involved in the uptake of long-chain |
| | | | fatty acids |
| T3 | Podoviridae | Escherichia coli | Glucosyl-α-1,3-glucose terminus of |
| | | | rough LPS |
| T4 | Myoviridae | Escherichia coli | Protein O-8 (OmpC) with LPS |
| | | K-12 | |
| | | Escherichia coli B | Glucosyl-α-1,3-glucose terminus of |
| | | | rough LPS |
| T5 | Siphoviridae | Escherichia coli | Polymannose sequence in the |
| | | | O-antigen and protein FhuA |
| T6 | Myoviridae | Escherichia coli | Outer membrane protein Tax |
| | | | (involved in nucleoside uptake) |
| T7 | Podoviridae | Escherichia coli | LPS^{c} |
| U3 | Microviridae | Escherichia coli | Terminal galactose residue in LPS |
| λ | Siphoviridae | Escherichia coli | Protein LamB |
| φX174 | Microviridae | Escherichia coli | Terminal galactose in the core |
| | | | aligosaccharide of rough LPS |
| φ80 | Siphoviridae | Escherichia coli | Proteins FhuA and TonB^{b} |
| PM2 | Carticoviridae | Pseudoalteromonas | Sugar moieties on the cell surface^{d} |
| E79 | Myoviridae | Pseudomonas | Core polysaccharide of LPS |
| | | aeruginosa | |
| jG004 | Myoviridae | Pseudomonas | LPS |
| | | aeruginosa | |
| φCTX | Myoviridae | Pseudomonas | Core polysaccharide of LPS, with |
| | | aeruginosa | emphasis on L-rhamnose and |
| | | | D-glucose residues in the outer core |
| φPLS27 | Podoviridae | Pseudomonas | Galactosamine-alanine region of the |
| | | aeruginosa | LPS core |
| φ13 | Cystoviridae | Pseudomonas | Truncated O-chain of LPS |
| | | syringae | |
| ES18 | Siphoviridae | Salmonella | Protein FhuA |
| Gifsy-1 | Siphoviridae | Salmonella | Protein OmpC |
| Gifsy-2 | | | |
| SPC3S | Siphoviridae | Salmonella | BtuB as the main receptor and |
| | | | O12-antigen as adsorption-assisting |
| | | | apparatus |
| SPN1S | Podoviridae | Salmonella | O-antigen of LPS |
| SPN2TCW | | | |
| SPN4B | | | |
| SPN6TCW | | | |
| SPN8TCW | | | |
| SPN9TCW | | | |
| SPN13U | | | |
| SPN7C | Siphoviridae | Salmonella | Protein BtuB |
| SPN9C | | | |
| SPN10H | | | |
| SPN12C | | | |
| SPN14 | | | |
| SPN17T | | | |
| SPN18 | | | |
| [symbol] | Myoviridae | Salmonella | Protein OmpC |
| S16 | | | |
| (S16) | | | |
| L-413C | Myoviridae | Yersinia pestis | Terminal GlcNAc residue of the LPS |
| P2 vir1 | | | outer core. HepII/HepIII and HepI/Glc |
| | | | residues are also involved in receptor |
| | | | activity* |
| ϕ1A1 | Myoviridae | Yersinia pestis | Kdo/Ko pairs of inner core residues. |
| | | | LPS outer and inner core sugars are |
| | | | also involved in receptor activity* |
| [symbol] | Podoviridae | Yersinia pestis | HepI/Glc pairs of inner core residues. |
| [symbol] | | | HepII/HepIII and Kdo/Ko pairs are also |
| | | | involved in receptor activity* |
| Pokrovskaya | Podoviridae | Yersenia pestis | HepII/HepIII pairs of inner core |
| YepE2 | | | residues. HepI/Glc residues are also |
| YpP-G | | | involved in receptor activity* |
| ϕA1122 | Podoviridae | Yersenia pestis | Kdo/Ko pairs of inner core residues. |
| | | | HepI/Glc residues are also involved in |
| | | | receptor activity* |
| PST | Myoviridae | Yersenia | HepII/HepIII pairs of inner core |
| | | pseudotuberculosis | residues* |

(b) Receptors in the O-chain structure that are enzymatically cleaved by phages

| | | | |
|---|---|---|---|
| ΩH | Podoviridae | Escherichia coli | The α-1,3 mannosyl linkages between |
| | | | the triaccharide repeating unit |
| | | | α-mannosyl-1,2-α-mannosyl-1,2- |
| | | | mannose |
| c341 | Podoviridae | Salmonella | The O-acetyl group in the mannosyl- |
| | | | rhamnosyl-O-acetylgalactose |
| | | | repeating sequence |
| P22 | Podoviridae | Salmonella | α-Rhmanosyl 1-3 galactose linkage of |
| | | | the G-chain |
| [symbol] | Podoviridae | Salmonella | [-β-Gal-Man-Rha-] polysaccharide |
| | | | units of the O-antigen |
| Sf6 | Podoviridae | Shigella | Rha II 1-α-3 Rha III linkage of the |
| | | | O-polysaccharide |

(a) Receptors in flagella

| | | | |
|---|---|---|---|
| SPN2T | Siphoviridae | Salmonella | Flagellin protein FHC |
| SPN3C | | | |
| SPN8T | | | |
| SPN9T | | | |
| SPN11T | | | |
| SPN13B | | | |
| SPN16C | | | |
| SPN45 | Siphoviridae | Salmonella | [symbol] |
| [symbol] | | | |
| [symbol] | | | |
| [symbol] | | | |
| SPN19 | | | |
| [symbol] | Siphoviridae | Salmonella | [symbol] |

(b) Receptors in pull and mating pair formations structures

| | | | |
|---|---|---|---|
| [symbol] | Siphoviridae | [symbol] | [symbol] |
| [symbol] | | | |
| Fd | | Escherichia coli | [symbol] |
| Pf | | | |
| f3 | | | |
| M13 | | | |
| PSD1 | [symbol] | Escherichia coli | Mating pair formation (Mpf) complex in |
| | | | the membrane |
| [symbol] | [symbol] | [symbol] | [symbol] |
| MPK7 | Podoviridae | [symbol] | [symbol] |
| [symbol] | Siphoviridae | [symbol] | [symbol] |
| [symbol] | Siphoviridae | [symbol] | [symbol] |

(c) Receptors in bacterial capsules

| 25 | Podoviridae | Escherichia coli | [symbol] | | |
|---|---|---|---|---|---|
| K11 | Podoviridae | [symbol] | [symbol] | | |
| [symbol] | Myoviridae | Salmonella | [symbol] | | |
| [symbol] | Siphoviridae | Salmonella | [symbol] | | |
| [symbol] | Podoviridae | Salmonella | [symbol] | | |
| [symbol] | | | | | |
| [symbol] | | | | | |
| [symbol] | | | | | |
| [symbol] | | | | | |
| | | Genus/ | | Primary | Secondary |
| Bactoeriphage | Family | group | Host | receptor | receptor |
| T1 | S | T1-like | E. coli | ? | FhuA (requires |
| | | | | | TonB) |
| T4 | M | T4-like | E. coli, Shigella | OmpC | LPS core |
| T5 | S | T5-like | E. coli | LPS O-antigen (polyman- | FhuA |
| | | | | nose)-optionally | |
| BF23 | S | T5-like | E. coli | LPS? | BtuB |
| λ | S | lambdoids | E. coli | OmpC | LamB |
| | | (λ-like) | | | |
| P22 | P | lambdoids | E. coli | LPS O-antigen | LPS? |
| | | (P22-like) | | | |
| Sf6 | P | ? | Shigella flexneri | LPS | OmpA, |
| | | | | | OmpC |
| N4 | P | N4-like | E. coli | ? | NfrA |
| G7C | P | N4-like | E. coli 4s | LPS O-antigen O22-like | unknown |
| | | | | | (OmpA and ?) |
| Alt63 | P | N4-like | E. coli 4s | LPS O-antigen | unknown |
| | | | | | (OmpA and ?) |
| CPS1 and | M | ? | Campylobacter jejuni | exopolysaccharide; | ? |
| related | | | NCTC12658 | modification of the | |
| phages | | | | MeOPN type is important | |
| | | | | for some phages | |
| CP220 and | M | ? | Campylobacter jejuni | motile flagellum | ? |
| related | | | NCTC12658 | | |
| phages | | | | | |
| NCTC12673 | | | Campylobacter jejuni | glycosylated flagellin | ? |
| VP5 | ? | ? | Vibrio cholerae | ? | OmpW |
| | | | O1 El Tor | | |
| phiR1-37 | ? | ? | Yersinia similis O9 | LPS O-antigen | ? |
| | | | and other Yersinia | | |
| SSU5 | S | | Salmonella enterica, | LPS external core | ? |
| | | | Shigella, E. coli K-12 | | |
| S16 | M | T4-like | Salmonella | OmpC | ? |
| VP4 | | | Vibrio cholerae | LPS O-antigen | ? |
| | | | O1 El Tor | | |
| phiX216 | M | P2-like | Burkholderia mallei | LPS O-antigen | ? |
| | | | B. pseudomallei | of B. mallei | |
| SPC35 | S | T5-like | Salmonella enterica | LPS O-antigen | BtuB |
| | | | serovar Typhimurium | | |
| SPN10H | S | T5-like | S. enterica serovar | LPS? | BtuB |
| (and 6 other | | | Typhimurium | | |
| isolates) | | | | | |
| SPN2T (and | S | ? | S. enterica serovar | flagellum | ? |
| 10 other | | | Typhimurium | | |
| isolates) | | | | | |
| SPN1S (and | P | ? | S. enterica serovar | LPS | ? |
| 6 other | | | Typhimurium | | |
| isolates) | | | | | |
| phiA1122 | P | T7-like | Yersinia pestis, | ? | Hep/Glc- |
| | | | Y. pseudotuberculosis | | Kdo/Ko |
| | | | | | regions of |
| | | | | | LPS core |
| phiCb13 and | S | ? | Caulobacter | flagellum | pili portal |
| phiCbK | | | crescenius | | |
| Mlol | S | ? | Mesorhizobium loti | LPS | LPS (?) |
| ST27, ST29, | ? | unknown | S. enterica serovar | ? | TolC |
| ST35 (and | | | Typhimurium | | |
| probably 14 | | | | | |
| more unchar- | | | | | |
| acterized | | | | | |
| phages) | | | | | |
| IMM-01 | S | ? | enterotoxigenic E. | ? | CS7 |
| | | | coli (ETEC) | | colonization |
| | | | | | factor (pilus) |
| VP3 | P | T7-like | V. cholerae O1 El Tor | LPS core | |
| EPS7 | S | T5-like | S. enterica, E. coli | ? | BtuB |
| 37 isolates of | ? | lambdoids | E. coli (?) | ? | FhuA |
| lambdoid | | | | | |
| phages from | | | | | |
| feces | | | | | |
| HS | S | T5-like | S. enterica serovar | ? | BtuB |
| | | | Enteritidis | | |
| OJ367 | ? | ? | Salmonella derby | ? | 45 kDa Omp |
| DMS3 | S | ? | Pseudomonas | ? | type IV pili |
| | | | aeruginosa | | |
| TLS | M | T-even | E.coli | TolC ? | TolC ? |
| Gifsy1, | ? | ? | S. enterica var. | ? | OmpC |
| Gifsy2 | | | Typhimurium | | |
| K139 | ? | Kappa | V. cholerae O1 El Tor | LPS O-antigen | ? |
| K20 | M | T-even | E. coli | OmpF and LPS core | OmpF and |
| | | | | | LPS core |
| phiCr30 | S | ? | C. crescentus | RsaA 130K protein | ? |
| | | | | of S-layer | |
| AP50 | Tect. | ? | Bacillus anthracis | Sap protein of S-layer | ? |
| CNRZ | M | ? | Lactobacillus | SlpH protein of S-layer | ? |
| 832-B1 | | | helveticus | | |
| SPP1 | S | SPP1 | Bacillus subtilis | glycosylated poly(Gro-P) | YueB |
| | | | | teichoic acids of the cell wall | |
| A118, P35 | S | | Lysteria | serovar-specific teichoic | ? |
| | | | monocytogenes | acids of the cell wall | |

[symbol]

indicates data missing or illegible when filed

The present invention provides for a method for screening for gene function for a bacteriophage, the method comprising: (a) providing one or more DNA barcoded overexpression strain libraries (such as Dub-seq) using DNA of the host organism and/or phage, and (b) screening for gain-of-function (GOF).

In some embodiments, the providing one or more DNA barcoded overexpression strain libraries using DNA of the host organism and/or phage comprises cloning a partial or total host/phage genome DNA fragments into a library of barcoded vector, such as a vector that can stably reside in the host organism, wherein each resulting vector comprises a host/phage genome DNA fragment integrated into the vector, such as using the method taught in Example 1, wherein the host organism(s) can be any host organism, such as any described in Table 1.

In some embodiments, where needed, the providing step comprises end repairing the fragments, phosphorylating the repaired fragments, and ligating the phosphorylated repaired fragments to the vector.

In some embodiments, the screening step comprises transforming a phage library into cloning bacterial strain, such as an E. coli strain, collecting the transformants, growing to saturation, and characterizing barcoded junctions derived from the phage library.

In some embodiments, the DNA fragments, or at least about 50%, 60%, 70%, 70%, 80%, or 90% DNA fragments, have an average size of from about 1.0 kilobasepairs (kbp), 1.5 kbp, 2.0 kbp, 2.5 kbp, 3.0 kbp, 3.5 kbp, 4.0 kbp, 4.5 kbp, 5.0 kbp, 5.5 kbp, or 6.0 kbp, or an average size within the range of any two preceding values. In some embodiments, the DNA fragments, or at least about 50%, 60%, 70%, 70%, 80%, or 90% DNA fragments, have sizes that fall within a range of any two of the following values: about 1.0 kbp, 1.5 kbp, 2.0 kbp, 2.5 kbp, 3.0 kbp, 3.5 kbp, 4.0 kbp, 4.5 kbp, 5.0 kbp, 5.5 kbp, and 6.0 kbp. In some embodiments, the vector is a medium copy vector.

In some embodiments, the providing one or more DNA barcoded overexpression strain libraries using DNA of the host organism and/or phage comprises shearing genomes of one or more bacteriophages inserting a barcoded transposon into a host organism, such as using the method taught in Example 1, wherein the bacteriophages(s) can be any bacteriophages(s) which correspond to a single host, such as any described in Table 1.

In some embodiments, there is one species of host organism and a plurality of bacteriophage species wherein each bacteriophage species is capable of infecting the host organism. In other embodiments, there are a plurality of host organism species and one bacteriophage species wherein the bacteriophage species is capable of infecting each host organism species in the plurality of host organism species.

In some embodiments, the functions comprise one or more of the following: recognition, entry, replication, and host lysis.

Both technologies employ a high-throughput DNA barcode sequencing readout (BarSeq) that enable cost effective and genome-wide assays of gene fitness in a single-pot assay.

In some embodiments, each barcode is a barcode taught in U.S. Patent Applications Pub. No. 2018/0030435, hereby incorporated by reference in its entirety.

In some embodiments, the providing and/or screening steps are automated and/or high throughout. In some embodiments, each individual host organism and/or phage sample is provided and/or screened in a format configured for automated and/or high throughout processing and/or handling, such as a 96-well format.

With increasing antibiotic resistance instances, there is urgent need for practical targeted alternatives to treat infection in humans, animals, water, fisheries and the entire food cycle. Phages are considered as possible alternatives because of their ready availability against any bacteria, specificity of interaction, smaller genomes, and their harmless growth cycle to human/animal host. Indeed, there are multiple instances of use of phages successfully to treat infection in humans, animals, water, fisheries, or the like. There is a need for methods to identify, track and quantify therapeutic phages in diverse application areas, and currently there are no such reported methods. The invention disclosed herein includes a method to barcode phages without compromising their host bacteria killing activity and growth cycle, and provide an avenue to identify, track, and quantify known therapeutic phages

Phages have smaller genomes compared to bacteria. So far, there are not reports on systematic loss-of-function (LOF) libraries of phages, wherein each gene is deleted and impact of that loss of gene studied on phage infection cycle. Phage genomes do not have a single region that is common and conserved across all phages/bacterial viruses. This creates a challenge to identify a region that is not essential for phage growth and infection. With advancement of mutant library creation by RB-Tnseq method or CRISPR-Cas system use, this barrier of studying gene-essentiality can be overcome, and then by using standard or state of the art molecular biology and genetic approaches, these phages/bacterial viruses can be uniquely barcoded with randomized DNA region.

The present invention provides for a LOF library of phages using available technologies such as RB-Tnseq or CRISPR-Cas system to study gene essentiality and then use the non-essential gene location to insert a unique “n-mer DNA barcode”. Here the non-essential gene does not impact the infectivity of a phage. The barcode comprises an n-mer randomized or defend DNA region surrounded by primer binding region that helps in amplifying the ‘barcode’. This barcoding strategy will create a handle for identifying, quantifying, and tracking a barcoded phage. By barcoding the wild-type phage isolated from nature, this will protect the effort and investment went into isolating the biological agent.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Schematic of ‘Phage foundry’: Integrated platform to generate comprehensive genome-wide libraries for diverse hosts and phages, perform functional fitness screens with diverse phages, fitness screen for anti-Cas9 factors and producing viral reagents to drive studies in microbial community manipulation with the goal of supporting various agricultural, environmental, health and biomanufacturing strategies.

FIG. 2. Preliminary dataset on T7 phage-E. coli interaction determinants; Selected genes with fitness scores shown as a heatmap for E. coli BW25113 RBTnseq and Dubseq libraries. Yellow color on the heatmap is for more fit strain and blue is for less fit strain in presence of T7 phage. LPS biosynthetic pathway shown with top hits in blue when deleted, and red (rcsA) when overexpressed.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “barcode” or “barcodes” can refer to nucleic acid codes or sequences associated with a target within a sample. A barcode can be, for example, a nucleic acid label. A barcode can be an entirely or partially amplifiable barcode. A barcode can be entirely or partially sequenceable barcode. A barcode can be a portion of a native nucleic acid that is identifiable as distinct. A barcode can be a known sequence. A barcode can be a random sequence. A barcode can comprise a junction of nucleic acid sequences, for example a junction of a native and non-native sequence. As used herein, the term “barcode” can be used interchangeably with the terms, “index”, “tag,” or “label-tag.” Barcodes can convey information. For example, in various embodiments, barcodes can be used to determine an identity of a nucleic acid, a source of a nucleic acid, an identity of a cell, and/or a target.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA.

A nucleic acid can comprise one or more analogs (e.g. altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone of the nucleic acid can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,′:4,5)pyrrolo[2,3-d]pyrimidin-2-one).

Methods of Quantitative Analysis of Nucleic Acid Target Molecules

Some embodiments disclosed herein provide methods of constructing an expression library from a plurality of nucleic acid fragments. In some embodiments, the plurality of nucleic acid fragments are from a single cell, a plurality of cells, a tissue sample, a virus, a fungus, or any combination thereof. The nucleic acid fragments can be DNA, such as genomic DNA, cDNA, and the likes; or RNA, such as mRNA, microRNA, tRNA, rRNA, and the likes. In some embodiments, the plurality of nucleic acid fragments can be a plurality of genomic fragments. In some embodiments, the plurality of genomic fragments can comprise a completely or partially sequenced genome, a single cell genome, a viral genome, a bacterial genome, a metagenome, or any combination thereof. In some embodiments, the plurality of nucleic acid fragments are from a single cell, a plurality of cells, a tissue sample, a virus, a fungus, or any combination thereof. The nucleic acid fragments can have a variety of sizes. For example, the plurality of nucleic acid fragments can have an average size that is, is about, is less than, is greater than, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, or a range between any two of the above values. In some embodiments, the nucleic acid fragments can be obtained by a fragmenting treatment, including but not limited to enzymatic treatment such as restriction enzyme digestion, physical treatment such as sonication, etc.

In some embodiments, the methods comprise providing a plurality of vectors. In some embodiments, each vector comprises one or more barcodes. The plurality of vectors can comprise at least about 100, 1,000, 10,000, 100,000, 1,000,000, or more vectors. In some embodiments, each vector comprises two barcodes. The barcode, or the two barcodes, can be selected from a set of unique barcodes. The barcode or the two barcodes can be completely random in sequence which can be sequenced before (or after) nucleic acid fragment cloning. In some embodiments, the plurality of vectors can be characterized so that each vector is identified with a unique barcode or a unique combination of two or more barcodes. In some embodiments, the characterization of the vectors comprises sequencing at least a portion of the one or more barcodes. In some embodiments, the two barcodes in a vector are next to each other. In some embodiments, the two barcodes are separated by one or more restriction sites. In some embodiments, the two barcodes are separated by one or more selection marker genes.

A barcode can comprise a nucleic acid sequence that provides identifying information for the specific nucleic acid fragment associated with the barcode. A barcode can be at least about 1, 2, 3, 4, 5, 6, 7, 8, 9, 10, 11, 12, 13, 14, 15, 20, 25, 30, 35, 40, 45, 50, or more nucleotides in length. A barcode can be at most about 300, 200, 100, 90, 80, 70, 60, 50, 40, 30, 20, 15, 12, 10, 9, 8, 7, 6, 5, 4, or fewer nucleotides in length. In some embodiments, there may be as many as 10⁶ or more different barcodes in the set of unique barcodes. In some embodiments, there may be as many as 10⁵ or more different barcodes in the set of unique barcodes. In some embodiments, there can be as many as 10⁴ or more different barcodes in the set of unique barcodes. In some embodiments, there can be as many as 10³ or more different barcodes in the set of unique barcodes. In some embodiments, there can be as many as 10² or more different barcodes in the set of unique barcodes.

In some embodiments, a barcode can be flanked by a pair of binding sites for two universal primers. The two universal primers can be the same or different. In some embodiments, each barcode of the plurality of vectors is flanked by the same pair of binding sites.

An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, a virus, a recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. The vector can be a variety of suitable replication units, including but not limited to: plasmids, viral vectors, cosmids, fosmids, and artificial chromosomes. In some embodiments, the vector is a broad-host-range replication vector. For example, there are a wide range of broad-host plasmids, cosmids and fosmids available based on IncQ, IncW, IncP, and pBBR1-based systems that can replicate in diverse microbes (Lale et al., (2011) Broad-host-range plasmid vectors for gene expression in bacteria. Strain engineering: Methods and protocols (Ed., James Williams), Methods in molecular biology, Vol 756, Chapter 19, 327-343).

In some embodiments, the vector can comprise a promoter sequence, such as a constitutive promoter, a synthetic promoter, an inducible promoter, an endogenous promoter, an exogenous promoter, or any combination thereof. In some embodiments, the vector can comprise a poly-A sequence. In some embodiments, the vector can comprise a translation termination sequence, and/or a transcription termination sequence. In some embodiments, the vector can further encode a tag sequence.

In some embodiments, the methods comprise inserting the plurality of nucleic acid fragments into the plurality of vectors to generate a plurality of expression vectors. In some embodiments, the plurality of nucleic acid fragments can be ligated with one or more adaptors before inserting into the vectors. In some embodiments, the one or more adaptors comprise one or more barcodes and/or one or more binding sites for a universal primer. A barcode alone, or two barcodes in combination, can be associated with the nucleic acid fragment that is inserted into the vector. For example, the nucleic acid fragment inserted into the vector can be flanked by the two barcodes.

Inserting the nucleic acid fragments can comprise ligation, such as blunt end ligation. In some embodiments, the vectors can be digested with a restriction enzyme to linearize the vectors. In some embodiments, the linearized vectors are blunt-ended before the ligation with the nucleic acid fragments.

In some embodiments, the methods comprise transforming the plurality of expression vectors into a host organism. A host organism is a bacterial cell. In some embodiments, the methods comprise growing the transformed host organism under a selection condition, so that only the host organisms transformed with the expression vector can survive. In some embodiments, the bacterial cells are or comprise Gram-negative cells, and in some embodiments, the bacterial cells are or comprise Gram-positive cells. Examples of bacterial cells of the invention include, without limitation, Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus Enterococcus faecalis, Bacillus coagulans, Bacillus ceretus, Bacillus popillae, Synechocystis strain PCC6803, Bacillus liquefaciens, Pyrococcus abyssi Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis.

In some embodiments, the host organism is one or more hosts described in Table 1 herein, and the bacteriophage is one or more bacteriophages described in Table 1 which correspond to the host.

With rapid rise in instances of antibiotic resistant bacteria and other deleterious effects caused by antibiotics on commensal healthy microbiome, there is an increased awareness to find novel solutions to antibiotics. One proposed alternative is to use bacterial viruses or bacteriophages that prey and kill pathogenic bacteria. However, decades of research has shown that bacteria use a spectrum of strategies to protect themselves from phage infection. These interaction studies between bacteria and phages have been largely performed on few key model bacterium/phage strains. Even in well studied model systems, we still do not know the full breadth of host resistance mechanisms to diverse phages. To realize the widespread successful practice of phage therapy, we need to know the phage resistance mechanisms and understand factors important in host infection pathways. Unfortunately, the current methods used to detect phage receptors suffer from tedious sample preparations, expensive sequencing methods and low throughout assays. We need new technologies that are quantitative, scalable, economical, can be applied to diverse hosts and phages at different multiplicity of infection. Such genome-wide approaches for identifying these phage-host interaction determinants would be highly valuable for obtaining systems-level understanding of phage infection pathways and phage-resistance phenotypes ands such approaches are necessary to develop phage-based strategies for precise microbial community engineering. In addition, by knowing phage receptors, it would be possible in the future to make rationally designed cocktails of phages that target different host pathways and eliminate the possibility of phage resistance.

Two genetic technologies enable fast and effective genome-wide screens for gene function, and are suitable for discovering host genes crucial in phage infection. The first, randomly barcoded transposon sequencing (RB-TnSeq) method, generates strain libraries for screening loss-of-function mutant phenotypes. The second method generates DNA barcoded overexpression strain libraries (Dub-seq) method using DNA of the host or phage and permits gain-of-function assays. Both technologies employ a high-throughput DNA barcode sequencing readout (BarSeq) that enable cost effective and genome-wide assays of gene fitness in a single-pot assay. These method decouple the genetic characterization from phenotype determination steps, and enable the entire pipeline of characterization cheaper, quantitative, less laborious and scalable than any currently available technologies. This disclosure details on invention of doing high throughput screens to discover phage receptors and other host factors that are important in phage infection and resistance. These competitive fitness assays can also be used for screening and discovering resistance factors for phage-like bacteriocins, bacterial predators, antimicrobial peptides and enzymes.

These method decouple the genetic characterization from phenotype determination steps, and enable the entire pipeline of characterization cheaper, quantitative, less laborious and scalable than any currently available technologies. For these two loss-of-function and gain-of-function screens to work, we had to optimize the multiplicity of infection, time of assay, sample preparation and data analysis pipelines.

Our combination of loss-of-function and gain of function methods enable researchers to gain mechanistic insights into antimicrobial compounds, phages, and phage like particles. This enables in designing rational cocktail formulation. Currently this is done in a very ad hoc fashion and subjected to lot of failures.

### REFERENCES CITED

1 Alivisatos, A. P. et a. MICROBIOME. A unified initiative to harness Earth's microbiomes. Science 350, 507-508, doi:10.1126/science.aac8480 (2015).
2 Blaser, M. J. et al. Toward a Predictive Understanding of Earth's Microbiomes to Address 21st Century Challenges. MBio 7, doi:10.1128/mBio.00714-16 (2016).
3 Clemente, J. C., Ursell, L. K., Parfrey, L. W. & Knight, R. The impact of the gut microbiota on human health: an integrative view. Cell 148, 1258-1270, doi:10.1016/j.cell.2012.01.035 (2012).
4 Buchan, A., LeCleir, G. R., Gulvik, C. A. & Gonzalez. J. M. Master recyclers: features and functions of bacteria associated with phytoplankton blooms. Nat Rev Microbiol 12. 686-698. doi:10.1038/nrmicro3326 (2014).
5 Philippot, L., Raaijmakers, J. M., Lemanceau, P. & van der Putten, W. H. Going back to the roots: the microbial ecology of the rhizosphere. Nat Rev Microbiol 11, 789-799. doi:10.1038/nrmicro3109 (2013).
6 Mendes, R., Garbeva, P. & Raaijmakers, J. M. The rhizosphere microbiome: significance of plant beneficial, plant pathogenic, and human pathogenic microorganisms. FEMS Microbiol Rev 37, 634-663, doi:10.1111/1574-6976.12028 (2013).
7 Biteen, J. S. et al. Tools for the Microbiome: Nano and Beyond. ACS Nano 10. 6-37, doi:10.1021/acsnano.5b07826 (2016).
8 Woloszynek, S. et al. Engineering Human Microbiota: Influencing Cellular and Community Dynamics for Therapeutic Applications. Int Rev Cell Mol Biol 324, 67-124. doi:10.1016/bs.ircmb.2016.01.003(2016).
9 Sheth, R. U., Cabral, V., Chen, S. P. & Wang, H. H. Manipulating Bacterial Communities by in situ Microbiome Engineering. Trends Genet 32, 189-200, doi:10.1016/j.tig.2016.01.005 (2016).
10 Mueller, U. G. & Sachs. J. L. Engineering Microbiomes to Improve Plant and Animal Health. Trends Microbiol 23, 606-617, doi:10.1016/j.tim.2015.07.009 (2015).
11 Guo. L. et al. Precision-guided antimicrobial peptide as a targeted modulator of human microbial ecology. Proc Nat Acad Sci USA 112, 7569-7574, doi:10.1073/pnas.1506207112(2015).
12 Abeles, S. R. & Pride. D. T. Molecular bases and role of viruses in the human microbiome. J Mol Biol 426, 3892-3906, doi:10.1016/j.jmb.2014.07.002 (2014).
13 Cadwell, K. The virome in host health and disease. Immunity 42. 805-813, doi:10.1016/j.immuni.2015.05.003(2015).
14 Breitbart, M. Marine viruses: truth or dare. Ann Rev Mar Sci 4, 425-448, doi:10.1146/annurev-marine-120709-142805 (2012).
15 Suttle, C. A. Marine viruses—major players in the global ecosystem. Nat Rev Microbiol 5, 801-812, doi:10.1038/nrmicro1750 (2007).
16 Brum, J. R. & Sullivan, M. B. Rising to the challenge: accelerated pace of discovery transforms marine virology. Nat Rev Microbiol 13, 147-159, doi:10.1038/nrmicro3404 (2015).
17 Roux, S., Hallam, S. J., Woyke. T. & Sullivan, M. B. Viral dark matter and virus-host interactions resolved from publicly available microbial genomes. Elife 4. doi:10.7554/eLife.08490(2015).
18 Roucourt, B. & Lavigne, R. The role of interactions between phage and bacterial proteins within the infected cell: a diverse and puzzling interactome. Environ Microbiol 11, 2789-2805, doi:10.1111/j.1462-2920.2009.02029.x (2009).
19 Lu, T. K. & Koeris, M. S. The next generation of bacteriophage therapy. Curr Opin Microbiol 14, 524-531. doi:10.1016/j.mib.2011.07.028 (2011).
20 Citorik, R. J., Mimee, M. & Lu. T. K. Bacteriophage-based synthetic biology for the study of infectious diseases. Curr Opin Microbiol 19, 59-69, doi:10.1016/j.mib.2014.05.022 (2014).
21 Frampton, R. A., Pitman, A. R. & Fineran. P. C. Advances in bacteriophage-mediated control of plant pathogens. Int J Microbiol 2012, 326452, doi:10.1155/2012/326452 (2012).
22 Koskella, B. & Meaden, S. Understanding bacteriophage specificity in natural microbial communities. Viruses 5, 806-823, doi:10.3390/v5030806 (2013).
23 Bruder, K. et al. Freshwater Metaviromics and Bacteriophages: A Current Assessment of the State of the Art in Relation to Bioinformatic Challenges. Evol Bioinform Online 12, 25-33, doi:10.4137/EBO.S38549 (2016).
24 Pires, D. P., Cleto, S., Sillankorva. S., Azeredo. J. & Lu, T. K. Genetically Engineered Phages: a Review of Advances over the Last Decade. Microbiol Mol Biol Rev 80, 523-543. doi:10.1128/MMBR.00069-15 (2016).
25 Nobrega, F. L., Costa, A. R., Kluskens, L. D. & Azeredo, J. Revisiting phage therapy: new applications for old resources. Trends Microbiol 23.185-191, doi:10.1016/j.tim.2015.01.006 (2015).
26 Kutter, E. et al. Phage therapy in clinical practice: treatment of human infections. Curr Pharm Biotechnol 11, 69-86 (2010).
27 Balogh. B., Jones, J. B., Iriarte, F. B. & Momol, M. T. Phage therapy for plant disease control. Curr Pharm Biotechnol 11, 48-57 (2010).
28 Hagens, S. & Loessner. M. J. Bacteriophage for biocontrol of foodborne pathogens: calculations and considerations. Curr Pharm Biotechnol 11, 58-68 (2010).
29 Wetmore, K. M. et al. Rapid quantification of mutant fitness in diverse bacteria by sequencing randomly bar-coded transposons. MBio 6, e00306-00315, doi:10.1128/mBio.00306-15(2015).
30 Mutalik V K et al. Characterization of functional traits using dual barcoded shotgun expression library sequencing. (in preparation) (2017).
31 Dy, R. L., Richter, C., Salmond. G. P. & Fineran, P. C. Remarkable Mechanisms in Microbes to Resist Phage Infections. Annu Rev Virol 1, 307-331. doi:10.1146/annurev-virology-031413-085500 (2014).
32 Labrie, S. J., Samson, J. E. & Moineau, S. Bacteriophage resistance mechanisms. Nat Rev Microbiol 8. 317-327, doi:10.1038/nrmicro2315 (2010).
33 Samson, J. E., Magadan, A. H., Sabri, M. & Moineau, S. Revenge of the phages: defeating bacterial defences. Nat Rev Microbiol 11, 675-687, doi:10.1038/nrmicro3096 (2013).
34 Diaz-Munoz, S. L. & Koskella, B. Bacteria-phage interactions in natural environments. Adv Appl Microbial 89, 135-183, doi:10.1016B978-0-12-800259-9.00004-4 (2014).
35 Seed, K. D. Battling Phages: How Bacteria Defend against Viral Attack. PLoS Pathog 11. e1004847, doi:10.1371/journal.ppat.1004847 (2015).
36 Qimron, U., Marintcheva, B., Tabor, S. & Richardson, C. C. Genomewide screens for Escherichia coli genes affecting growth of T7 bacteriophage. Proc Natl Acad Sci USA 103, 19039-19044, doi:10.1073/pnas.0609428103 (2006).
37 Christen, M. et al. Quantitative Selection Analysis of Bacteriophage phiCbK Susceptibility in Caulobacter crescentus. J Mol Biol 428, 419-430, doi:10.1016/j.jmb.2015.11.018 (2016).
38 Maynard, N. D. et al. A forward-genetic screen and dynamic analysis of lambda phage host-dependencies reveals an extensive interaction network and a new anti-viral strategy. PLoS Genet 6, e1001017. doi:10.1371/journal.pgen.1001017 (2010).
39 De Smet, J., Hendrix, H., Blasdel, B. G., Danis-Wodarczyk. K. & Lavigne. R. Pseudomonas predators: understanding and exploiting phage-host interactions. Nat Rev Microbiol, doi:10.1038/nrmicro.2017.61(2017).
40 Ando, H., Lemire, S., Pires, D. P. & Lu, T. K. Engineering Modular Viral Scaffolds for Targeted Bacterial Population Editing. Cell Syst 1, 187-196, doi:10.1016/j.cels.2015.08.013 (2015).
41 Lu. T. K. & Collins. J. J. Engineered bacteriophage targeting gene networks as adjuvants for antibiotic therapy. Proc Natl Acad Sci USA 106, 4629-4634, doi:10.1073/pnas.0800442106(2009).
42 Robinson. D. G., Chen, W., Storey, J. D. & Gresham, D. Design and analysis of Bar-seq experiments. G3 (Bethesda) 4, 11-18, doi:10.1534/g3.113.008565 (2014).
43 Smith, A. M. et al. Quantitative phenotyping via deep barcode sequencing. Genome Res 19, 1836-1842, doi:10.1101/gr.093955.109 (2009).
44 M. N. Price et al. Deep Annotation of Protein Function across Diverse Bacteria from Mutant Phenotypes. bioRxiv, doi: 10.1101/072470 (2017).
45 Xu, Y. et a. Bacteriophage therapy against Enterobacteriaceae. Virol Sin 30, 11-18, doi:10.1007/s12250-014-3543-6 (2015).
46 Summers, W. C. Bacteriophage research: early research. In: E. Kutter and A. Suiakvelidze (eds.). Bacteriophages: Biology and Application. CRC Press, Boca Raton, Fla., 5-28 (2005).
47 Abedon, S. T. The murky origin of Snow White and her T-even dwarfs. Genetics 155. 481-486(2000).
48 R. Calendar and S. T. Abedon (eds.). The Bacteriophages. Oxford University Press, Oxford. 2 ed.,
49 Miller, E. S. et al. Bacteriophage T4 Genome. Microbiology and Molecular Biology Reviews 67, 86-156, doi:10.1128/mmbr.67.1.86-156.2003 (2003).
50 Grose, J. H. & Casjens. S. R. Understanding the enormous diversity of bacteriophages: the tailed phages that infect the bacterial family Enterobacteriaceae. Virology 468-470, 421-443, doi:10.1016/j.virol.2014.08.024 (2014).
51 de Moraes, M. H. et al. Salmonella Persistence in Tomatoes Requires a Distinct Set of Metabolic Functions Identified by Transposon Insertion Sequencing. Appl Environ Microbiol 83, doi:10.1128/AEM.03028-16 (2017).
52 Whichard, J. M. et al. Complete genomic sequence of bacteriophage felix o1. Viruses 2, 710-730, doi:10.3390/v2030710 (2010).
53 Marti, R. et al. Long tail fibres of the novel broad-host-range T-even bacteriophage S16 specifically recognize Salmonella OmpC. Mol Microbiol 87, 818-834, doi:10.1111/mmi.12134(2013).
54 Silby, M. W., Winstanley, C., Godfrey, S. A., Levy. S. B. & Jackson, R. W. Pseudomonas genomes: diverse and adaptable. FEMS Microbiol Rev 35, 652-680, doi:10.1111/j.1574-6976.2011.00269.x (2011).
55 Ganeshan, G. & Manoj Kumar, A. Pseudomonas fluorescens, a potential bacterial antagonist to control plant diseases. Journal of Plant Interactions 1, 123-134, doi:10.1080/17429140600907043(2005).
56 Haas, D. & Defago, G. Biological control of soil-borne pathogens by fluorescent pseudomonads. Nat Rev Microbiol 3, 307-319, doi:10.1038/nrmicro1129 (2005).
57 Hol, W. H., Bezemer, T. M. & Biere, A. Getting the ecology into interactions between plants and the plant growth-promoting bacterium Pseudomonas fluorescens. Front Plant Sci 4, 81, doi:10.3389/fpls.2013.00081 (2013).
58 Preston, G. M. Plant perceptions of plant growth-promoting Pseudomonas. Philos Trans R Soc Lond B Biol Sci 359. 907-918, doi:10.1098/rstb.2003.1384 (2004).
59 Frampton, R. A. et al. Genome, Proteome and Structure of a T7-Like Bacteriophage of the Kiwifruit Canker Phytopathogen Pseudomonas syringae pv. actinidiae. Viruses 7. 3361-3379, doi:10.3390/v7072776 (2015).
60 Box. A. M., McGuffie, M. J., O'Hara. B. J. & Seed, K. D. Functional Analysis of Bacteriophage Immunity through a Type I-E CRISPR-Cas System in Vibrio cholerae and Its Application in Bacteriophage Genome Engineering. J Bacteriol 198, 578-590, doi:10.1128/JB.00747-15(2015).
61 Seed, K. D., Lazinski, D. W., Calderwood, S. B. & Camilli, A. A bacteriophage encodes its own CRISPR/Cas adaptive response to evade host innate immunity. Nature 494, 489-491, doi:10.1038/nature11927 (2013).
62 Gonzalez-Garcia. V. A. et al. Characterization of the initial steps in the T7 DNA ejection process. Bacteriophage 5, e1056904, doi:10.1080121597081.2015.1056904 (2015).
63 Carlson, K. Working with bacteriophages: Common techniques and methodological approaches. In. Kutter E, Sulakvelidze A. editors. Bacteriophages biology and applications. Boca Raton, Fla.: CRC Press. pp. 437-494 ((2004)).
64 Dulbecco, R. Mutual exclusion between related phages. J Bacteriol 63, 209-217 (1952).
65 Abedon, S. T. Bacteriophage secondary infection. Virol Sin 30. 3-10, doi:10.1007/s12250-014-3547-2 (2015).
66 Anderson. C. W. & Eigner, J. Breakdown and exclusion of superinfecting T-even bacteriophage in Escherichia coli. J Virol 8.869-886 (1971).
67 Lu, M. J. & Henning. U. Superinfection exclusion by T-even-type coliphages. Trends Mircrobiol 2, 137-139 (1994).
68 McAllister, W. T. & Barrett. C. L. Superinfection exclusion by bacteriophage T7. J Virol 24, 709-711 (1977).
69 Barrangou, R. & van der Oost, J. Bacteriophage exclusion, a new defense system. EMBO J 34, 134-135. doi:10.15252/embj.201490620 (2015).
70 Bondy-Denomy, J. et al. Prophages mediate defense against phage infection through diverse mechanisms. ISME J 10, 2854-2866, doi:10.1038/ismej.2016.79 (2016).
71 Lu, M. J. & Henning. U. The immunity (imm) gene of Escherichia coli bacteriophage T4. J Virol 63, 3472-3478 (1989).
72 Decker, K., Krauel, V., Meesmann, A. & Heller, K. J. Lytic conversion of Escherichia coli by bacteriophage T5: blocking of the FhuA receptor protein by a lipoprotein expressed early during infection. Mol Microbiol 12, 321-332 (1994).
73 Hofer. B., Ruge. M. & Dreiseikelmann, B. The superinfection exclusion gene (sieA) of bacteriophage P22: identification and overexpression of the gene and localization of the gene product. J Bacteriol 177, 3080-3088 (1995).
74 Fogg. P. C., Allison. H. E., Saunders, J. R. & McCarthy, A. J. Bacteriophage lambda: a paradigm revisited. J Virol 84, 6876-6879, doi:10.1128/JVI.02177-09 (2010).
75 Bertani, G. & Deho. G. Bacteriophage P2: recombination in the superinfection preprophage state and under replication control by phage P4. Mol Genet Genomics 266, 406-416, doi:10.1007/s004380100527 (2001).
76 Cumby, N., Edwards. A. M., Davidson, A. R. & Maxwell, K. L. The bacteriophage HK97 gp15 moron element encodes a novel superinfection exclusion protein. J Bacteriol 194, 5012-5019, doi:10.1128/JB.00843-12 (2012).
77 Nesper, J., Blass, J., Fountoulakis, M. & Reidl, J. Characterization of the major control region of Vibrio cholerae bacteriophage K139: immunity. exclusion, and integration. J Bacteriol 181, 2902-2913 (1999).
78 Doudna, J. A. & Charpentier, E. Genome editing. The new frontier of genome engineering with CRISPR-Cas9. Science 346, 1258096, doi:10.1126/science.1258096 (2014).
79 Carter, J., Hoffman, C. & Wiedenheft, B. The Interfaces of Genetic Conflict Are Hot Spots for Innovation. Cell 168, 9-11. doi:10.1016/j.cell.2016.12.007 (2017).
80 Pawluk, A. et al. Naturally Occurring Off-Switches for CRISPR-Cas9. Cell 167, 1829-1838 e1829, doi:10.1016/j.cell.2016.11.017 (2016).
81 Rauch, B. J. et al. Inhibition of CRISPR-Cas9 with Bacteriophage Proteins. Cell 168, 150-158 e110, doi:10.1016/j.cell.2016.12.009 (2017).
82 Bondy-Denomy. J., Pawluk, A., Maxwell, K. L. & Davidson, A. R. Bacteriophage genes that inactivate the CRISPR/Cas bacterial immune system. Nature 493. 429-432. doi:10.1038/nature11723 (2013).
83 Qi, L. S. et al. Repurposing CRISPR as an RNA-guided platform for sequence-specific control of gene expression. Cell 152. 1173-1183, doi:10.1016/j.cell.2013.02.022 (2013).
84 Kiro, R., Shitrit, D. & Qimron, U. Efficient engineering of a bacteriophage genome using the type I-E CRISPR-Cas system. RNA Biol 11, 42-44, doi:10.4161/rna.27766 (2014).
85 Paez-Espino, D. at al. Uncovering Earth's virome. Nature 536, 425-430, doi:10.1038/nature19094(2016).
86 Mahony. J., Ainsworth, S., Stockdale, S. & van Sinderen, D. Phages of lactic acid bacteria: the role of genetics in understanding phage-host interactions and their co-evolutionary processes. Virology 434, 143-150, doi:10.1016j.virol.2012.10.008 (2012).
87 Marco, M. B., Moineau, S. & Quiberoni, A. Bacteriophages and dairy fermentations. Bacteriophage 2, 149-158, doi:10.4161/bact.21868 (2012).
88 Chubiz. L. M., Lee, M. C., Delaney. N. F. & Marx, C. J. FREQ-Seq: a rapid, cost-effective, sequencing-based method to determine allele frequencies directly from mixed populations. PLoS One 7, e47959, doi:10.1371/journal.pone.0047959 (2012).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Discovery and Engineering of Host-Phage Interaction Determinants for Designed Manipulation of Microbial Communities

Microbial communities drive and are driven by significant environmental processes, affect agricultural output, and impact human and animal health^{1,2}. Complex interactions among themselves, their hosts and environments are thought to be important for these effects¹⁻⁶. Manipulation of these communities can potentially lead to improved health, crop productivity and environmental resilience⁷⁻¹¹. The virome—the collection of viruses that parasitize these microbial communities—are a critical feature of microbial community dynamics, activity and adaptation^{4,12,13}.

Though viruses/phages represent the most abundant biological entities with an estimated range of 1030-1032-tenfold greater than bacteria^{14,15}, the virome is deeply under-characterized, which limits our ability to understand microbial community dynamics and activity or to utilize this resource for microbial community-based interventions^{12,16-22}. For example, 114 of the 278 genes of one of the best-studied model viruses Enterobacteriophage T4 are currently annotated as hypothetical in GenBank²³. Since phage encode relatively small genomes they are inherently engineerable at genome-scale and there is an opportunity to gain control of bacteriophage to “edit” the behaviors of individual members of microbial communities in situ to obtain understanding and targeted applications^{9,20,24}. Indeed, trials have been run using engineered/evolved phage cocktails to clear pathogens in agriculture, in food industry, in animals and humans^{19,25-28}.

We aim to develop a platform to gain a deeper understanding of phage-host interaction and phage engineering, and we demonstrate the power of this platform by application to a targeted set of important phages and their hosts. Success of this project will enable us to rapidly characterize phages, phage resistance determinants of the host and apply the knowledge to phage engineering to selectively manipulate or edit individual members of microbial communities that impact plant productivity and animal/human health. To uncover host factors important for phage infection and resistance, we will employ two recently developed technologies in our laboratories that enable fast and quantitative genome-wide screens for gene function. Specifically, we will use the RB-Tnseq²⁹ (randomly barcoded transposon sequencing) method, to generate strain libraries for screening loss-of-function mutant phenotypes and the Dubseq³⁰ (dual barcoded Shotgun expression library sequencing) method for screening gain-of-function phenotypes. We will employ these technologies to create strain libraries and study host-phage interaction determinants for a diverse class of double-stranded DNA phages against Escherichia coli, Salmonella enterica, Pseudomonas fluorescence, Pseudomonas syringae and Vibrio cholerae, which represent phylogenetically similar, commensal and pathogenic strains found in the normal flora of plants, animals and humans. To gain deeper understanding of host/phage defense mechanisms, to study superinfection mechanisms and to discover novel anti-CRISPR factors, we will build and screen Dubseq library of phage genomes in respective hosts. Finally, we will apply these foundational studies in formulating design principles for engineering phage particles and employing them for microbial community manipulations.

B. Project Description:

a. Relevance and Justification

Bacteria use a spectrum of strategies to protect themselves from phage infection. Some of these strategies include phage adsorption inhibition, blocking DNA entry, restriction-modification systems, toxin-antitoxin systems and CRISPR-Cas systems³¹⁻³⁵. However, the mechanisms of these phage-host interaction strategies have been largely derived from focused studies on a handful of individual bacterium/phage systems. It has been realized that genome-wide approaches for identifying these phage-host interaction determinants would be highly valuable for obtaining systems-level understanding of phage infection pathways and phage-resistance phenotypes³⁶⁻³⁸ and we are in need of methods that are easily transferable to new systems. Such approaches are necessary to develop phage-based strategies for precise microbial community engineering³⁹. Indeed, a number of studies have highlighted the importance of high-throughput technologies applied to phage engineering, genome assembly and significance of uncovering host-specificity determinants for further phage engineering applications^{9,24,39-41}.

However important, the currently used genome-wide screening methods to discover phage-host interaction determinants are very low throughput methods, labor intensive, less quantitative and cannot be scaled to assay tens of phages at different multiplicity of infection for a number of hosts under variable conditions^{36,37}. Recently, we have developed two genetic technologies that enable fast and effective genome-wide screens for gene function, and are suitable for discovering host genes crucial in phage infection. The first, randomly barcoded transposon sequencing (RBTnseq)²⁹, generates strain libraries for screening loss-of-function mutant phenotypes in nonessential genes. The second method generates DNA barcoded overexpression strain libraries (Dubseq)³⁰ using genome fragments of the host or that of the phage and permits gain-of-function assays in pooled competitive fashion.

Both technologies employ the same high throughput DNA barcode sequencing readout (Barseq) that enables cost effective, less-laborious, quantitative genomewide assays of gene fitness in a single-pot across diverse conditions^{29,42,43}. As an example of efficiency, we have been able to apply RB-Tnseq across 32 diverse bacteria in over 4800 genomewide condition assays to make 18.7 million gene phenotype measurements in just over a couple of years44. We expect similar scaling for the related Dubseq technology.

Here, we propose to develop a characterization platform to uncover molecular determinants of phage-host interaction and phage engineering, and we demonstrate the power of this platform by applying it to a targeted set of important phages and their hosts. In this 3-year project, we will focus on elucidating the host-phage interaction networks in key Gammaproteobacteria hosts: Escherichia coli and Salmonella enterica; Pseudomonas fluorescens, Pseudomonas syringae, & Vibrio cholerae that occur in diverse forms in nature, ranging from commensal strains in the normal flora to those pathogenic to plants, humans or animal hosts. We will uncover host and phage molecular determinants of bacteriophage specificity & resistance mechanisms of the isolated members of the community using high-throughput functional genomics and use the resulting data to engineer phage with specificity against a single species in a synthetic microbial community or deliver engineered host strains resistant to a class of phage.

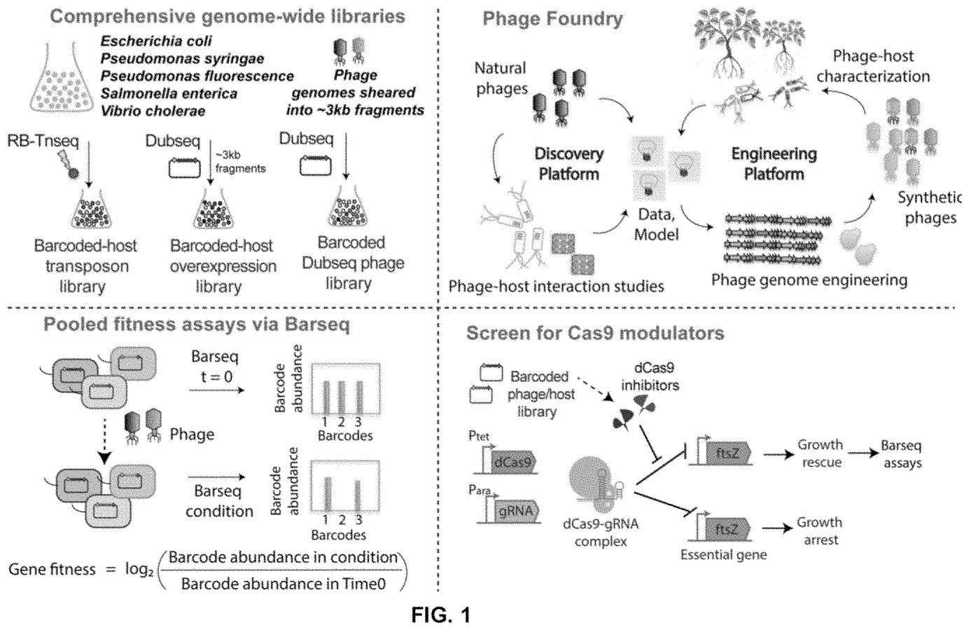

Success of this project will lay the foundation of a ‘Phage foundry’ (FIG. 1), which will provide knowledge and viral reagents to the broad research community and can be focused to support the agricultural, environmental and health strategies of IGI's academic and industrial partners. By developing the foundational knowledge and genome-engineering platform to enable precise microbiome manipulations this project aligns rightly with IGI's mission statement to treat diseases and to improve food safety.

b. Research Plan:

There are two main goals of this three-year research proposal. For the first two years of the project we will implement tools and assays essential for meeting goal 1 tasks.

Goal 1: Uncovering Host-Bacteriophage Interaction Networks

To investigate phage-host interactions we will initially focus on E. coli and its double-stranded DNA phages for which there is a sizable amount of published work that can be used to interpret and validate the results. We will use existing E. coli K-12 loss-of-function (LOF) libraries (RB-Tnseq) and gain-of function (GOF) libraries (Dubseq), to determine the diverse host factors that impact the infectivity of E. coli phages. We will extend these forward genetic methods to other E. coli strains (E. coli BL21, E. coli C, E. coli NCTC12900), plant associated bacteria P. fluorescence and P. syringae, as well as the animal/human pathogens Salmonella enterica servoar Typhi and Vibrio cholerae by creating LOF and GOF libraries in each strain to study the phage-interaction determinants.

1.1 Phage Resistance Mechanisms

Background: E. coli and its phages: Verotoxigenic E. coli is a leading cause of millions of infections each year and causes many human deaths in developing countries (CDC.gov/ecoli). Persistence in plants, agriculture produce and water represents an important life cycle for this pathogen, and bacteriophages have been proposed as biocontrol agents^{28,45}. Even-though, here we will be studying phage-host interaction determinants using nonpathogenic and nontoxigenic E. coli (BW25113, BL21, E. coli C, E. coli NCTC12900) these studies are valuable in gaining understanding of pathogenic E. coli. Our exploration of these diverse E. coli strains will also give us insight into how much phage resistance mechanisms vary nature and phage effectiveness as hosts vary. Since early efforts to focus phage research to a small group of ‘authorized phages’ designated as T-phages, an extensive body of research has been carried out on these E. coli Type 1-Type 7 (T1 to T7) phages^{46,47} and have been milestones in the development of molecular biology field. These phages are known to use overlapping but distinct mechanisms of host recognition, entry, replication and lysis 4. However, the host genes necessary for phage infection pathway have not been completely identified, more than half of phage genes still have no function assigned and most of host-phage interaction insights have come from multiple disparate studies^{48,49}. Two recent studies employed genome-wide approaches to elucidate molecular determinants of T7 phage36 and lambda phage infection of E. coli³⁸. While these studies did discover new host genes playing a key role in the phage resistance, they were laborious, not scalable to hundreds of assays (across different phage titers) and hard to extend to other hosts and viruses. Our RB-Tnseq and Dubseq platforms use a simple, scalable barcode-sequencing assay termed Barseq^{29,42,43} and enable largescale investigation of gene phenotypes in single-pot assays. We have access to diverse E. coli phages including T-phages (T2, T3, T4, T5, T6, T7 phages), N4 phage, 186 phage, Lambda cI857 phage, P2 phage and less well studied T-like phages (LZ4 phage, CEV1 and CEV2 phages) in addition to T7 phage mutants and T4 phage mutants that lack multiple nonessential genes. The E. coli RB-Tnseq and Dubseq libraries enable systematic genome-wide studies of these phages at different phage titers. Such an endeavor will yield a valuable data detailing general phage infectivity pathways and phage resistant mechanisms. By screening such canonical phages against different E. coli strains will improve our understanding of the different receptors recognized by different phages, their cross-talk, different host factors important in phage infection and how these results differ between strains because of their genotype.

In addition to E. coli and its phages, we have considered four medically/industrially important organisms and their phages: plant associated bacteria P. fluorescence, plant pathogen P. syringae, and animal/human pathogens Salmonella enterica serovar Typhi and Vibrio cholerae. These model organisms are amenable to our high-throughput genetic technologies and assay system and represent a good diversity in gammaproteobacteria and bacteriophage phylogeny50. A brief background on each of these hosts and their phages is presented below.

Salmonella and its phages: Salmonella enterica subspecies enterica serovar Typhimurium LT2 is a facultative pathogen that causes numerous infections, including typhoid fever, gastroenteritis, and septicemia (cdc.gov/Salmonella). Recently, it is also becoming persistent colonizer of animals, plants, fruits and vegetables, and causing millions of non-typhoid salmonellosis infections leading many human deaths per year51. We have access to four key Salmonella phages: Felix O1, T7-like SP6 phage, T4-like S16 phage and P22. Among these, Felix O1 is known to recognize diverse Salmonella and hence has been used in diagnosing Salmonella in food samples and agriculture produce⁵². Similarly, recently discovered S16 shows broad Salmonella recognition53. P22 phage is well known molecular biology tool for transduction, while SP6 phage known to recognize LPS as E. coli T7 phage48. Each of these phages has been topic of detailed study, but none have been subject of genome-wide screens. Any insights into how these phages interact with their host would be a valuable because of their applicability in diagnostic and phage therapy.

Pseudomonas and its phages: The Pseudomonas genus is one of the versatile groups of bacteria that are plant commensal (P. fluorescence), plant pathogen (P. syringae), animal and human pathogen (P. aeruginosa), and bioremediation specialist (P. putida)^{39,54}. Here we will be focusing on P. fluorescence and P. syringae, and their phages. P. fluorescence has been known to improve plant growth via nutrient cycling, pathogen antagonism and induction of plant defenses⁵⁵⁻⁵⁸ while P. syringae is known to infect numerous economically important plants, fruits and vegetables⁵⁴. Phage therapy has been proposed as one of the biocontrol measures and a tool to manipulate microbial community around rhizosphere^{27,39,59}. We have access to a number of Pseudomonas phages namely Phi2, PhiIBB-PF7A infecting P. fluorescence and our collaborator Britt Koskella has FRS, FTP, M5.1, WILS and J120 phages that infect P. syringae. The receptor for most of these phages is not known and none of these phages have been subjected to genome-wide screens for studying host recognition and resistance. Detailed understanding of host-phage determinants will enable rational phage engineering and microbiome manipulations.

Vibrio cholerae and its phages: Vibrio cholerae serogroup O1 is water-borne pathogen, which causes Cholera epidemics and leads to thousands of human deaths each year (cdc.gov/cholerae). Cholera spreads through contaminated water and there is an unmet need for clinical intervention for stopping the spread of the deadly disease (http://www.who.int/cholera/en/). Different lytic phages have been isolated from stools of cholera patients and may be involved in easing the disease burden⁶⁰. ICP1 is the most dominant phage, has T4 like morphology, and a set of them have been shown to encode their own CRISPR-Case system that they use to adaptively evade host defenses⁶¹. Our collaborator Kim Seed has >20 isolates of this phage from clinical samples collected 2011-2017. We also have access to ICP3 a T7 like phage, and many isolates of ICP2 phage whose genome is unique. ICP1 and ICP2 recognize LPS 01 antigen and OmpU porin respectively⁶⁰. The receptor for ICP3 is not yet known. Detailed insights about the host recognition, phage receptor and infection pathway for each of these phages would be highly valuable for devising rational phage cocktails.

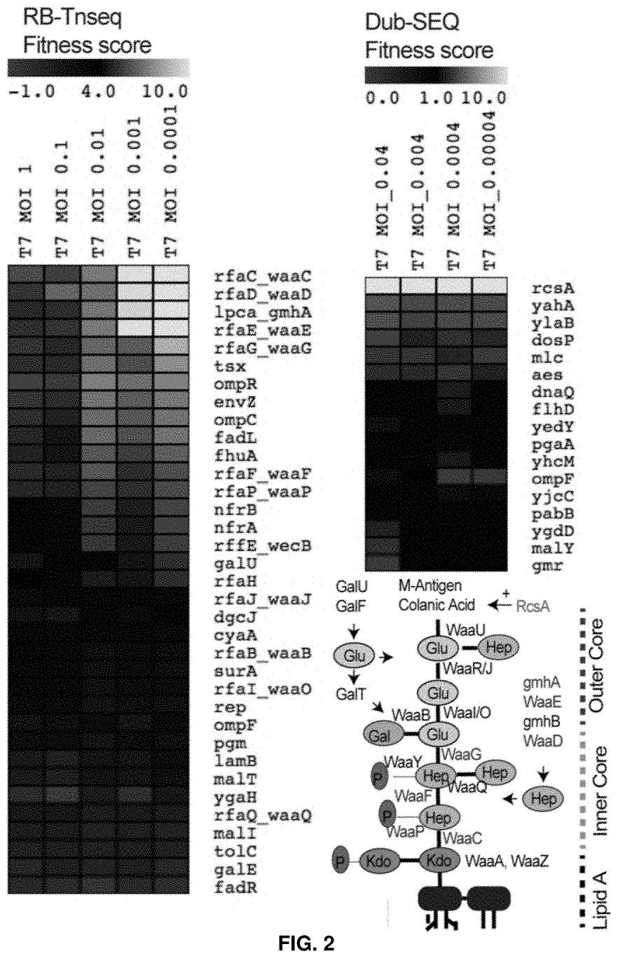

Preliminary studies: As a proof-of-principle demonstration of our methodology, we used in-house built E. coli LOF and GOF libraries and performed competitive fitness assays in presence of increasing titers of T7 phage per bacterial cell (MOI or multiplicity of infection). E. coli LOF strains were created by insertion of a barcoded transposon in E. coli BW25113 (for RBTnseq) and GOF strains were created by cloning E. coli BW25113 DNA fragments of ˜3 kbp into a medium copy barcoded broad-host plasmid. Both methods rely on the use of random 20 nucleotide DNA barcodes (one barcode in the case of RB-Tnseq and two barcodes in the case of Dubseq) and one time Illumina sequencing for characterizing initial library mapping using a Tnseq-like protocol. We challenged both RB-Tnseq and Dubseq libraries to different MOI of T7 in planktonic cultures as well as top-agar based assay. We collected host library samples before and after 18 hrs of growth, extracted genomic DNA (in the case of RB-Tnseq) and plasmid DNA (in the case of Dubseq) from these samples and strain quantification was performed using a Barseq. For each experiment, every gene has an associated fitness score, defined as the log 2 ratio of abundance of that strain in the starting pool (T0) versus the abundance after the experiment run (Tcondition). Each experiment provided a quantitative, genome-wide view of genes that are necessary or detrimental to optimal fitness in presence of T7 phage (FIG. 2). For example, in the case of RB-Tnseq assay, we confirmed earlier observations that loss of E. coli genes involved in LPS biosynthesis severely affects T7 infectivity³⁶. It is known that LPS recognition by T7 phage is essential for its effective adsorption^{48,62}. The fitness data from Dubseq assays, agree with earlier observation that overexpression of resA gene (induces Colanic acid biosynthesis) inhibits T7 phage infection probably due to interference with phage receptor accessibility³⁶. This preliminary work established the assay methodology and broad applicability of RB-TnSeq and Dubseq for performing competitive pooled assays in presence of diverse class of phages. Using this approach, we can perform hundreds of genome-wide fitness experiments, in 48-well format, at reasonable cost. Up to 96 different fitness experiments can be multiplexed in a single lane of Illumina HiSeq 4000, at a cost of ˜$10 per assay. In the following section, we present our experimental plan on extending E. coli competitive fitness assays to different types of phages and E. coli strains, and other host-phage combinations.

Experimental plan: We have a diverse collection of E. coli phages, S. enterica phages, P. fluorescence phages, P. syringae phages and V. cholerae phages obtained from other labs and our collaborators. These serve as a great resource for performing fitness experiments across different hosts. We follow standard protocols for phage propagation, handling and storage⁶³. By using available E. coli BW25113 RB-Tnseq and Dubseq library, we will perform competitive fitness assays in presence of T2, T3, T4, T5, T6, N4, LZ4, CEV1, CEV2, Lambda cI857, P2,186 phage as described in the above section. To compare the phage infectivity pathway determinants across different E. coli strains, we will create LOF and GOF libraries in E. coli BL21, E. coli C and E. coli NCTC12900 (non-toxigenic O157:H7 strain). To generate LOF RB-Tnseq library, we will follow the published protocol29. Briefly, we will conjugate E. coli BL21, E. coli C and E. coli NCTC12900 with a pool of donor E. coli MW3064 carrying Tn5 or mariner transposon vector on LB agar supplemented with DAP. After 6 hours of conjugation, conjugants will be washed with sterile media to remove DAP, and plated on LB agar supplemented with kanamycin. After overnight incubation, kanamycin resistant colonies will be collected and regrown before making glycerol stocks. The genome preparation of this stock will be used to map the barcode insertion site on the genomic location using Tnseq methodology²⁹. To generate Dubseq library of E. coli BL21, E. coli C and E. coli NCTC12900, we will shear total genomic DNA to 3 kB of each host, end-repair and clone the DNA fragments between a pair of DNA barcodes on a vector derived from the broad host vector pBBR1MCS-2. We will build the library of 100,000 clones by transforming into E. coli DH10B. We will use a Tnseq-like Illumina sequencing protocol to map the DNA barcode identities to DNA fragments on the plasmid. Using this strategy, we will be able to map the exact breakpoints of each of the 100,000 clones and associate each with a pair of unique DNA barcode sequences. Once these associations are completed, we will transform the Dubseq library into E. coli BL21, E. coli C and E. coli NCTC12900 before proceeding to perform pooled competitive assays with different phages. The sample processing and data analysis will be performed as explained in the preliminary studies and published method²⁹. We will follow up significant hits through targeted deletion and overexpression of the genes identified and confirmation of the phenotype observed in bulk assay.

To extend these studies to the plant associated bacteria P. fluorescence and P. syringae, as well as the animal pathogens S. enterica and V cholerae, we will create RB-Tnseq and Dubseq libraries for each host as detailed above. The transposon vectors used for RB-Tnseq library and overexpression vector used for Dubseq library reliably function in these hosts (unpublished data). We will perform validation experiments to confirm the quality of these libraries before assaying them in presence of a number of their known phages.

Expected outcomes: Our two genome-wide screening approaches (RB-Tnseq and Dubseq) are apt for rapidly identifying phage-host relationship networks for different types of phages against the same host, and for different phage-host combinations all at one time. These experiments will reveal a core set of host genes that are conditionally essential for different phage propagation mechanisms. By comparing results across phage-host combinations we will determine conserved genetic determinants of phage specificity, resistance and propagation and as well as those that differentiate among strain, close clades and species. In summary, this work will be the first global survey of host genes essential for diverse phage propagation and will provide a rich dataset for deeper biological insights and bioinformatic analysis. These experiments will also yield a number of testable hypotheses on host specificity, resistance and will be verified by engineering of those phage variants in genome assembly platform (Goal 2).

1.2 Determinants of Superinfection Mechanism

Background: During early studies on phage genetics it was observed that presence of prophage or infection by one phage excludes infection by another phage during mixed infection⁶⁴. Such phenomenon, in which preexisting phage infection prevents a secondary infection by the same or different phage, is known as ‘superinfection exclusion⁶⁵⁻⁶⁸. Even though it has been hypothesized that this mechanism is widespread in diverse viruses, only few of superinfection exclusion systems are known to date^{67,69,70}. It appears that these genes or systems are encoded either on prophages or lytic phage genomes themselves, but how widespread these superinfection mechanisms in lytic phages and how they impact host fitness is less understood. Two well-studied examples for lytic bacteriophage are: E. coli phage T4 encodes two systems (Imm and Sp), which inhibit DNA injection of T4 and other T-even-like phages^{67,71}. T5 codes for L1p protein that is formed in preinfected cells and blocks its own receptor, thereby preventing superinfection by other T5 phages⁷². In addition to these lytic phages, superinfection exclusion systems are also reported for temperate prophages in S. enterica (bacteriophage P22)⁷³; E coli phages (Lambda)⁷⁴, (P2 phage)⁷⁵, (HK97 phage⁷⁶), V. cholerae (K139 bacteriophage)⁷⁷ and in a recent large scale characterization for P. aeruginosa prophages⁷⁰. Here, we will use Dubseq technology for creating phage overexpression libraries for E. coli, P. fluorescence, P. syringae, S. enterica and V. cholerae and screen for phage resistance phenotypes and underlying molecular determinants. These studies will yield design specification for phage engineering part of the project (Goal 2).

Experimental plan: To create phage Dubseq library for each host, we will sequence and pool phage genomes for each host, shear them to ˜3 Kb fragments, end-repair and clone them between dual barcodes on a broad-host vector system. The cloned fragments and associated barcodes will then be mapped to the genome via a Tnseq like protocol and subjected to pooled fitness assays in presence of different phages as described in section 1.1.

Expected outcome: This will be the first genome-wide study to discover different phage genes that exclude the infection of specific host by different phages there by identifying en masse superinfection exclusion systems. As phages are known to encode strongest promoters, some of the genome fragments may not get cloned in to our medium copy Dubseq vector due to host toxicity and may escape the characterization. Nevertheless, this first systematic attempt to discover diverse design principles causing exclusion mechanisms will be a valuable resource for phage engineering (Goal 2) and phage therapy applications.

1.3 Discovery of Anti-Cas9 Elements

Background: Since the discovery of Cas9, an RNA-guided DNA endonuclease enzyme from Streptococcus pyogenes associated with Clustered Regularly Interspersed Palindromic Repeats (CRISPR), can cleave both strands of complementary DNA target, the field of genome engineering has gone into a revolution mode⁷⁸. The precision genome editing technology via Cas9 is rapidly approaching clinical applications and discovery and engineering of diverse modes to regulate Cas9 activity are taking an important role⁷⁹. In this regard, a few recent efforts have used bioinformatics approaches successfully in identifying anti-CRISPR elements (Acrs for short) and showed that many of these Acr proteins bind directly to Cas9 and block its activity⁷⁹⁻⁸². We have been part of the initial work on developing applications for the catalytically inactive Cas9 system or dCas9 system⁸³ and have been working on implementing dCas9 genome-wide assays in diverse bacteria. We aim to use this technology in combination with Dubseq technology to screen for dCas9 modulators present on both host and phage genomes, and use insights from this study in developing phage engineering platform.

Experimental plan: We have an in-house developed dCas9 system for doing genome-wide knockdown assays in E. coli and we will use this system for screening dCas9 modulators. In this system, dCas9 is expressed from E. col chromosome and gRNA targeting essential ftsZ gene or chromosomally inserted mRFP gene is expressed from a high copy plasmid (FIG. 1). Induction of dCas9 and gRNA repressing ftsZ shuts down cellular growth, induction of gRNA repressing mRFP eliminates RFP expression. We will transform different phage Dubseq and host Dubseq libraries built in section 1.1 and 1.2 into E. coli carrying dCas9 assay system, and then induce dCas9 and gRNA expression to screen for strains that display either high mRFP expression (using flow cytometer) or growth (rescuing ftsZ knockdown). We will process the Dubseq plasmid preparation follow up the winning candidates by targeted experiments and uncover various modes of dCas9 interaction.

Expected outcome: Combination of phage and host Dubseq library technology with dCas9 assay system offers an unparalleled scale for discovering dCas9 modulators experimentally. The winning candidates from these experiments can then be used for in-depth bioinformatics search strategies for discovering additional modulators that might have missed in our experiments and early bioinformatics work. Finally, by identifying dCas9 modulators in our chosen set of hosts and their phages this work yields key design specifications for phage/host engineering.

Goal 2: Host Engineering, and Phage Genome Assembly and Engineering Platform for Microbial Community Manipulation

Background: Though phage encode relatively smaller genomes and are inherently ‘engineerable objects’, their in vitro genome assembly and modification has been low-throughout and laborious^{24,40,84}. A recently published yeast platform for assembling T7-like phage genomes seems to be promising technology for engineering diverse size phages40. There is an opportunity to design and assemble synthetic phages for gaining control of phage-host interactions, infectivity and to “edit” the behaviors of individual members of microbial communities in situ. One of the key challenges in this endeavor has been lack of characterization tools for phage-host interaction that can be sourced for designing phages for engineering applications^{40,85}. Results from Goal 1 will be able to fill this gap for diverse class of phages for the same strain or different strains using LOF and GOF libraries. In addition, data from a recent metagenomic study⁸⁵ can be sourced to engineer chimeric phage particles (for example, using tail fiber coding genes, genes coding for peptidoglycan-degrading enzymes, host-specific gRNA for CRISPR/Cas9 system or adhesion factors) and test their infection specificity and efficiency against specific hosts. Alternatively, data from Goal 1 will enable us to engineer hosts to be less susceptible to a particular phage as a way of providing “platform” strains that might be used industrially or therapeutically. Industrially, resistant hosts can be useful because of the bacterial contamination problem^{86,87}. In conceptual therapies, we might give beneficial or neutral engineered therapeutic microbes an advantage in the environment by making them resistant to endogenous or introduced phage that remove/predate non-beneficial members of the community, which they can otherwise ecologically replace⁹. In the second and third year of this project, we will apply the foundational knowledge generated from Goal 1 studies and a recent metagenomic study⁸⁵ in establishing design-build-test platform for phage engineering.

Experimental plan: To validate the technology⁴⁰, we will use PCR amplified overlapping fragments of E. coli phages and clone them in a yeast artificial chromosome (YAC) or a bacterial artificial chromosome (BAC) within yeast. To facilitate high-throughput pooled assays of multiple phage variants against a single host or microbial community, we will also use unique barcodes for each engineered/assembled phage variant. Recovery of the gap repaired-assembled YAC/BAC-phages from yeast followed by transformation into bacteria will yield active phage particles. These phage variants will be then tested for their host adsorption and plaque forming capability (specificity) with E. coli K-12 and B121 strains. Using this genome assembly platform, we will next generate diverse deletion and chimeric libraries of T7-like viruses that infect diverse Pseudomonads. In addition, we will engineer phage particles with a host-specific CRISPR/Cas9 system to selectively up-regulate or down-regulate a single essential gene in a single microbe in the synthetic microbial community.

As a proof-of-principle, we will use such engineered phage variants/cocktails to selectively eliminate a specific bacterium from a synthetic mixed population of different Pseudomonas and E. coli strains. We will employ an in-house optimized Freq-Seq method88 to quantify the outcome of phage treatment in the synthetic mixed population. Overall this project will give us an opportunity to set up an integrated discovery and engineering platform to produce viral reagents to drive studies of ‘plant and human-microbial community-phage’ interaction, and to support the agricultural, environmental and possibly health strategies of IGI collaborators.

### Example 2

### Methods to Barcode Phages to Identify, Track, Quantify and Protect Intellectual Property of Therapeutic Phages

In this invention, we use non-essential gene location of phage to insert a unique “n-mer DNA barcode” such that it may not impact the infectivity of a phage. These DNA barcodes are composed of n-mer randomized or defend DNA region surrounded by primer binding region that helps in amplifying the ‘barcode’. This barcoding strategy creates a handle for identifying, quantifying, and tracking a barcoded phage.

Methods

Plasmid Construction λ

A region encoding non-essential region in phage P1 genome (Lobocka et al., 2004) was selected for the insertion of DNA barcodes. 50 bp of the non-essential region was selected as the site for homologous recombination (Datsenko & Wanner, 2000, Piya et al., 2017). A DNA fragment consisting of the first 50 bp homology region of DNA, followed by a universal primer binding region (P1), followed by a 10-mer unique DNA barcode, a universal primer binding region (P2) and the last 50 bp homology region (FIG. 2) (Mutalik et al., 2019). This synthetic DNA was then cloned into a plasmid of choice for recombination step.

Barcode Insertion into Phage Genome

Phage λ Red proteins mediated homologous recombination was applied to insert DNA barcodes into phage P1 genome. Escherichia coli str. BioDesignER (Egbert et al., 2019) was used as the host in which the λ Red proteins are expressed from the genome. E. coli str. BioDesignER was transformed with the barcoded plasmid and the transformed strain was selected for antibiotic resistance. The transformed strain was then infected with phage P1 and lysates were harvested. The integration of DNA barcodes in P1 genome was verified via PCR with primers designed to bind to the binding region P1 and P2. To demonstrate we can retain the barcodes in phage cocktails, we inserted 2 different barcodes in phage P1, and then mixed with two lytic Coliphages T2 and T5. Essentially we have 2 phage cocktail formulations (P1-barcode1 with T2 and T5 phages; and P1-barcode2 with T2 and T5 phages). We used these phage formulations to study the growth curves of E. coli K-12 BW25113 strain growth. Both formulations efficiently inhibited bacterial growth. We used the lysates to genome prep the phage cocktail, and then performed PCR to amplify the barcodes with primers that enable sequencing on Illumina sequencing platforms. We employed in-house developed computational code to process the sequencing data, and quantified the barcodes. We performed these experiments in triplicates. These barseq PCR steps helped us to quantify and track P1 phages in both cocktail formulations.

### CONCLUSIONS

The results demonstrate the utility of this standardization approach in inserting genetic tags on phages. This phage barcoding simplifies tracking and quantification of phages in different contexts and makes the workflows economical, less laborious and is scalable to thousands of phages.

### REFERENCES CITED IN EXAMPLE 2

Adams, M. H., (1959) Bacteriophages. Interscience Publishers, New York, N. Y.
Block, S. M., Donoho, D., Hwa, T., Joyce, G., Nelson, D., Steams, T., Weinberger, P., and Williams, E. (2004) DNA Barcodes and Watermarks.
Datsenko, K. A., and Wanner, B. L. (2000) One-step inactivation of chromosomal genes in Escherichia coli K-12 using PCR products. Proc Natl Acad Sci USA 97: 6640-6645.
Dedrick, R. M., Guerrero-Bustamante, C. A., Garlena, R. A., Russell, D. A., Ford, K., Harris, K., Gilmour, K. C., Soothill, J., Jacobs-Sera, D., Schooley, R. T., Hatfull, G. F., and Spencer, H. (2019) Engineered bacteriophages for treatment of a patient with a disseminated drug-resistant Mycobacterium abscessus. Nat Med 25: 730-733.
Duyvejonck, H., Merabishvili, M., Pirnay, J. P., De Vos, D., Verbeken, G., Van Belleghem, J., Gryp, T., De Leenheer, J., Van der Borght, K., Van Simaey, L., Vermeulen, S., Van Mechelen, E., and Vaneechoutte, M. (2019) Development of a qPCR platform for quantification of the five bacteriophages within bacteriophage cocktail 2 (BFC2). Sci Rep 9: 13893.
Egbert, R. G., Rishi, H. S., Adler, B. A., McCormick, D. M., Toro, E., Gill, R. T., and Arkin, A. P. (2019) A versatile platform strain for high-fidelity multiplex genome editing. Nucleic Acids Res 47: 3244-3256.
Hesse, S., and Adhya, S. (2019) Phage Therapy in the Twenty-First Century: Facing the Decline of the Antibiotic Era; Is It Finally Time for the Age of the Phage? Annu Rev Microbiol 73: 155-174.
Lobocka, M. B., Rose, D. J., Plunkett, G., 3rd, Rusin, M., Samojedny, A., Lehnherr, H., Yarmolinsky, M. B., and Blattner, F. R. (2004) Genome of bacteriophage P1. J Bacteriol 186: 7032-7068.
McCallin, S., Sacher, J. C., Zheng, J., and Chan, B. K. (2019) Current State of Compassionate Phage Therapy. Viruses 11.
Mutalik, V. K., Novichkov, P. S., Price, M. N., Owens, T. K., Callaghan, M., Carim, S., Deutschbauer, A. M., and Arkin, A. P. (2019) Dual-barcoded shotgun expression library sequencing for high-throughput characterization of functional traits in bacteria. Nat Commun 10: 308.
Pires, D. P., Cleto, S., Sillankorva, S., Azeredo, J., and Lu, T. K. (2016) Genetically Engineered Phages: a Review of Advances over the Last Decade. Microbiol Mol Biol Rev 80: 523-543.
Piya, D., Vara, L., Russell, W. K., Young, R., and Gill, J. J. (2017) The multicomponent antirestriction system of phage P1 is linked to capsid morphogenesis. Mol Microbiol 105: 399-412.
Schmidt, C. (2019) Phage therapy's latest makeover. Nat Biotechnol 37: 581-586.
Schooley, R. T., Biswas, B., Gill, J. J., Hernandez-Morales, A., Lancaster, J., Lessor, L., Barr, J. J., Reed, S. L., Rohwer, F., Benler, S., Segall, A. M., Taplitz, R., Smith, D. M., Kerr, K., Kumaraswamy, M., Nizet, V., Lin, L., McCauley, M. D., Strathdee, S. A., Benson, C. A., Pope, R. K., Leroux, B. M., Picel, A. C., Mateczun, A. J., Cilwa, K. E., Regeimbal, J. M., Estrella, L. A., Wolfe, D. M., Henry, M. S., Quinones, J., Salka, S., Bishop-Lilly, K. A., Young, R., and Hamilton, T. (2017) Development and Use of Personalized Bacteriophage-Based Therapeutic Cocktails To Treat a Patient with a Disseminated Resistant Acinetobacter baumannii Infection. Antimicrob Agents Chemother 61.
Svircev, A., Roach, D., and Castle, A. (2018) Framing the Future with Bacteriophages in Agriculture. Viruses 10.
Todd, K. (2019) The Promising Viral Threat to Bacterial Resistance: the Uncertain Patentability of Phage Therapeutics and the Necessity of Alternative Incentives. Duke Law J 68: 767-805.
Ventola, C. L. (2015) The antibiotic resistance crisis: part 1: causes and threats. P T 40: 277-283.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A nucleic acid encoding a bacteriophage genome comprising a unique n-mer barcode inserted in a non-essential location or gene location within the bacteriophage genome, or a bacteriophage comprising the nucleic acid thereof.

2. The nucleic acid of claim 1, wherein the bacteriophage comprises a wild-type genome, except for the inserted unique n-mer barcode.

3. The nucleic acid of claim 1, wherein the n-mer DNA barcode inserted in a non-essential location or gene location does not interfere with the infection cycle of the bacteriophage, and/or does not compromise the lysis activity and/or growth cycle of a host bacterium infected by the bacteriophage. In some embodiments, the n-mer DNA barcode is flanked by a pair of primer binding regions that bind to a known pair of primers or a pair of primers of known nucleotide sequences, wherein the pair of primer binding regions facilitates the amplification of the n-mer barcode using the known pair of primers or the pair of primers of known nucleotide sequences.

4. A method of identifying the source or origin of a bacteriophage, the method comprising: (a) providing a sample comprises, or is suspected to comprise, a bacteriophage of claim 1; (b) amplifying the n-mer barcode using a known pair of primers or a pair of primers of known nucleotide sequences; (c) determining or identifying the nucleotide sequence of the n-mer barcode; and (d) correlating the n-mer barcode to a known nucleotide sequence which in turns correlates to an identity of a known bacteriophage; such that the source or origin of the bacteriophage is determined based on the correlation obtained in the correlating step.

5. The method of claim 4, wherein the providing step comprises obtaining the sample from a subject.

6. The method of claim 4, wherein the amplifying step comprises performing a polymerase chain reaction (PCR).

7. The method of claim 4, wherein the providing step is preceded by one or more of the following steps: constructing the bacteriophage by inserting a unique n-mer barcode into a wild-type bacteriophage, and/or releasing, administering, or selling or transferring the ownership of the bacteriophage, such as administering the bacteriophage to a subject suffering or suspected of suffering from a disease caused by a bacterium, which the bacteriophage is capable of infecting or is capable of being the host bacterium for the bacteriophage.

8. A library of bacteriophages wherein each bacteriophage comprises an insertion randomly inserted in the genome of the bacteriophage, such as at least part of the library comprising loss-of-function (LOF) bacteriophages, wherein optionally each bacteriophage comprises an n-mer barcode inserted in a non-essential gene location within the bacteriophage genome comprising loss-of-function (LOF), or a bacteriophage comprising the nucleic acid thereof.

9. The library of bacteriophages of claim 8, wherein the library is constructed using the RB-Tnseq or CRISPR-Cas system.

10. A method of determining the locations with a genome of a bacteriophage wherein the insertion of an n-mer barcode into the genome does not interfere with the infection cycle of the bacteriophage, and/or does not compromise the lysis activity and/or growth cycle of a host bacterium infected by the bacteriophage, the method comprises (a) constructing a library of LOF bacteriophages comprising an insertion randomly inserted the genome of the bacteriophage; (b) determining which bacteriophage is capable of infecting a host bacterium; (c) determining where on the genome of the bacteriophage the insertion is located; (d) inserting a unique n-mer barcode into the non-essential location or gene location identified in the bacteriophage to produce a barcoded bacteriophage; and (e) optionally administering the barcoded bacteriophage to a subject, such as a patient suffering from a disease caused by or infected with a host bacterium that the barcoded bacteriophage is capable of infecting.
